(19) United States
(12) Patent Application Publication
(10) Pub. No.: US 2024/0416016 A1
(43) Pub. Date: Dec. 19, 2024

(54) Internally Powered Breast Milk Collection Device

(71) Applicant: DAO Health, El Dorado Hills, CA (US)

(72) Inventors: Dan Garbez, El Dorado Hills, CA (US); Howard B. Rockman, Oakbrook Terrace, IL (US)

(73) Assignee: DAO Health, El Dorado Hills, CA (US)

(21) Appl. No.: 18/741,243

(22) Filed: Jun. 12, 2024

Related U.S. Application Data
(60) Provisional application No. 63/521,156, filed on Jun. 15, 2023.

(51) Int. Cl. A61M 1/06

(52) U.S. Cl. CPC A61M 1/064; A61M 1/067

(57) ABSTRACT

A breast milk collection device, comprising an adaptor adapted at a first wide end to receive a woman's breast and a second narrow end of the adaptor communicating with a reservoir. The reservoir is adapted to receive breast milk from the adaptor. The second narrow end of the adaptor has a first aperture and a hollow drip tube in communication with the first aperture. The hollow drip tube has a second aperture through which the hollow drip tube communicates with an internal volume of the reservoir. A one-way valve element communicates with the hollow drip tube and with the internal volume of the reservoir. A flexible barrier is disposed across a hollow chamber formed in a rigid barrier housing, the flexible barrier creating a hermetic seal between the rigid barrier housing and the hollow chamber, and the hollow chamber fluidly communicating with an interior of the hollow drip tube. A drive assembly including a reciprocally driven member contacts a first surface of the flexible barrier to reciprocally drive the flexible barrier to a first position during a first position of the reciprocally driven member. The flexible barrier returns to a second position during a second position of the reciprocally driven member, providing an alternating source of vacuum pressure and relief of vacuum pressure in the hollow drip tube.

This application claims priority to provisional application 63/521,156 filed Jun. 15, 2023, to the extent allowed by law.

### TECHNICAL FIELD

The present invention relates to the field of human breast milk collection devices and more specifically to breast milk collection devices that incorporate an internally located or partially internally and externally located mechanical movement system with respect to a reservoir, that cyclically applies and releases a vacuum force to a lactating woman's breast to express milk from the breast, the milk collected in the reservoir of the collection device and the entire device fitting discreetly within a woman's brassiere to allow hands free collection of the breast milk.

### BACKGROUND OF THE INVENTION

Breast milk collection devices for expressing and collecting breast milk from a lactating woman are well known in the art. However, the field of breast milk collection devices with self-contained, or removably integral reservoirs which can be used discreetly by fitting the breast pump within a woman's brassiere often under ordinary clothing so that a woman can use a breast pump around others discretely, is relatively new. Early devices upon which the present invention is an improvement are taught in U.S. Pat. Nos. 7,559,915 and 8,118,772 (Dao, Garbez), and U.S. Pat. Nos. 8,702,646 and 10,258,723 (Dao, Garbez, Paul, Sutton), all commonly assigned, the disclosures of which patents are incorporated by reference herein.

Other attempts to provide a breast milk collection device that fits discreetly within a woman's brassiere are offered by Momcozy (https://momcozy.com/products/momcozy-s9-pro-wearable-breast-pump); (https://momcozy.com/products/all-in-one-m5-wearable-breast-pump-painless-to-pump). Another breast milk collection device currently on the market is the Elvie Pump (www.elvie.com/en-us/shop/elvie-pump). Each of these breast milk collection devices, and many others currently being sold, include relatively large motor and vacuum pump assemblies as part of the overall structure of these devices, which vacuum pump assemblies protrude from a user's brassiere when used, preventing the woman from pumping and collecting milk in a public area discretely. Also, the reservoir volume in these breast milk collection devices is potentially decreased in a trade-off to make room for the motor and vacuum pump assembly. Breast milk can also be expressed, or released from the mother's lactating breasts, by massaging the breast by hand, or by application of traditional style manual or electromechanical pumping equipment acting upon the breasts that collect milk directly into baby bottles, both of which are commonly available in the domestic U.S. market.

Another breast milk collection device currently on the market is the Willow 360 wearable breast pump offered by Willow Innovations, Inc. https://shop.onewillow.com/collections/willow-gen-3). The Willow 360 wearable breast milk collection device also includes a large motor and vacuum pump assembly, which decreases the volume of the milk reservoir. Further, the Willow 360 wearable breast pump conveys expressed milk upward from a drip tube to the milk collection container, which requires added power to move the milk and introduces inefficiencies into the operation of the breast milk collection device.

Mothers who work outside the home must stop working approximately every two and a half hours to pump breast milk in order to maintain an adequate milk supply. When pumping and breast milk collection equipment is employed, it takes approximately 30 minutes for a mother to set up the equipment, undress, pump, collect the milk and perform cleanup. Because some breast milk pumping and collection systems require a mother to frontally undress, a private setting is usually deemed necessary. This process, which must be continually repeated every two and a half hours is isolating, cumbersome, and extremely disruptive during work.

Relatively low pressure, high volume, suction is required to pull the woman's breast into an adaptor of a breast milk collection device. A typical system that is cylinder-actuated operates as a closed system, trapping a volume of air into the adaptor. When the woman's breast is pressed in the adaptor, it seals itself against the sides of the adaptor and forms an enclosed space adjacent the nipple. When suction is applied, the malleable breast is pulled into the adaptor and toward the opening at the end of the funnel-shaped adaptor. A typical pump's cylinder, with an interior volume of several cubic inches, cycles back and forth repetitiously, completing an in-out “throw” over the course of a second or two, to create a massaging pulling rhythm upon the woman's breasts by alternating positive and negative pressure. This rhythm stimulates the mother's milk to be released, or “letdown,” whereupon it flows and is eventually collected in a reservoir of the breast milk collection device.

There are also a variety of other powered and manual breast milk collection devices available on the market that are well known in the relevant arts, ranging from very simple compact hand operated pumps, to innovative electromechanical concepts.

Certain pump assemblies for breast milk collection devices utilize an electromechanical construction with an impeller or other means that creates a constant suction with a pressure relief mechanism or valve, where the pump assembly builds up negative pressure to some predefined, preset or adjustable maximum, and then a relief valve or other means releases the negative pressure, so that during the cycle the vacuum pressure peaks, then is relieved and suction drops and approaches a more close-to-neutral negative pressure measurement. The negative pressure builds up, is relieved, and then the cycle repeats itself as the relief mechanism shuts itself off again and negative pressure begins to build up again.

Many breast pump assemblies during a typical cycle create negative pressure, and then alternately return toward a neutral pressure which may give the user the sense of an alternating negative and positive pressure, even though no positive pressure may actually be measured during the majority of cycles from the pump assembly. The adaptor, once placed against the breast, has a tendency to hold onto the breast, especially if the pump's cycle, once engaged and some air is purged from the trapped enclosure, alternates between strong negative pressure and weak negative pressure, but never alternates to all the way back to a neutral or positive pressure. If substantial positive pressure was actually introduced into this cycle, there would be a tendency to “blow” the adaptor off the breast, resulting in a disruption of the pumping rhythm, and possibly causing small amounts of milk in the adaptor to leak from the device and onto the user, rather than migrate into the milk collection container.

A secure seating of the adaptor to the breast is preferred to establish a consistent and relaxing rhythm which most mothers find essential to letting down their milk, which does not usually come for a minute or more after the pumping cycle has begun. The valve adapted for such pumps are usually weak enough so that simply the weight of expressed milk which builds up within the enclosure can force itself through the valve.

What is common to all pumps for breast milk collection devices, however, is that they of necessity stimulate the mother to release milk somewhat by replicating or approximating the sucking of an infant, and so the sensation that must be created by the pump assembly alternates between a somewhat strong negative pressure, and a weak or non-existent negative pressure, with a complete alternating cycle usually lasting only a second or two.

Pumping equipment that provides more negative pressure and a more reliable and consistent suction and rhythm provides the most benefit for mothers whose circumstances require that they must, for an extended period, frequently utilize a breast milk collection device.

U.S. Pat. No. 6,440,100 (Prentiss) presents a hands-free option which uses a low profile nipple cap held in place by a nursing brassiere. The nipple cap is placed over the nipple, and a tube, for both vacuum supply and milk collection, extends from below the nipple cap to a collection container. A vacuum source, such as an electric pump, draws the milk from both breasts into the collection container which hangs below the brassiere. While this solution goes a long way towards providing a hands-free design, the placement of the collection container outside of the brassiere is cumbersome and unwieldy when placing and removing this device.

Prentiss attempts to minimize the profile of the nipple cap by placing the vacuum source directly below the nipple. With this design, when suction is applied, the nipple is drawn at a downward angle, which tends to inhibit the flow and expression of milk by drawing the nipple onto the vacuum source or by pinching the milk ducts. Ideally, the nipple should be drawn forward to create the smooth and unobstructed action necessary to trigger the expression of milk. Elongation of the nipple and forward suction is the same as that applied by a suckling infant. The Prentiss design may result in the failure to trigger the milk expression reflex in many women.

It would be desirable to have a breast milk pumping and collection system that is hands free, and that is also easy to assemble, disassemble and clean, and reassemble, and to position fully under normal clothing without the need to undress or to don complicated and overtly visible harness systems.

A further need exists for a hands free, concealable and ergonomically shaped breast milk collection device that can be supported by an ordinary or nursing bra.

Another need exists for a breast milk collection device having a source of vacuum pressure applied to the breast to produce the expression of breast milk, wherein the source of vacuum pressure is totally isolated from the flow path of the milk from the breast into the collection device reservoir.

A further object of the present invention is to provide a wearable breast milk collection device wherein a low power relatively small mechanical movement is operatively connected to a flexible barrier to create an alternating source and relief of vacuum pressure in the breast milk collection device.

Yet another object of the present invention is to provide a low power relatively small mechanical movement operatively connected to a flexible barrier of a breast milk collection device, the mechanical movement and a power source located in and surrounded by a reservoir of the breast milk collection device. Alternately, the mechanical movement and power source is partially internally and externally located with respect to the reservoir of the breast milk collection device.

Another object of the present invention is to provide a breast milk collection device having a mechanical drive assembly, power source and control system, or portions thereof, detachable from the collection device, but outwardly configured into the form of the device that fits into the user's brassiere, while forming a single unit with the reservoir and the remaining elements of the breast milk collection device.

Consequently, a need exists for a fully operable breast milk collection device that fits completely within a woman's standard or nursing brassiere. Such a device would be less likely to interfere with breastfeeding from the opposite breast and avoid the isolating, disruptive and sometimes embarrassing need to disrobe to pump breast milk.

### SUMMARY OF THE INVENTION

The present invention is an improved breast milk collection device including an adaptor having a wide end to receive a woman's breast, and a second end of the adaptor communicating with a reservoir. The reservoir has an outer perimeter, and the reservoir receives breast milk expressed from the woman's breast through the adaptor. The adaptor has a narrow end and a first aperture at the narrow end, and a hollow drip tube communicates with the first aperture of the narrow end of the adaptor.

The hollow drip tube has a second aperture through which the hollow drip tube communicates with an internal volume of the reservoir. A one-way valve element communicates with the drip tube and with the internal volume of the reservoir, thus preventing fluid in a reservoir from backflowing into the drip tube.

In one embodiment, a flexible barrier extends across a hollow chamber formed in a rigid barrier housing to create a hermetic seal between a rigid barrier housing and the chamber formed in the rigid barrier housing. The hollow chamber fluidly communicates with the interior of the hollow drip tube. In an embodiment, the hollow chamber is formed at one end of the drip tube. In another embodiment, the hollow chamber is located away from the drip tube, but the hollow chamber fluidly communicates with the drip tube. The flexible barrier is capable of movement in the rigid barrier housing between first and second positions.

A physical drive assembly is located either within the outer perimeter of, or detachably adjacent to, the reservoir, and the physical drive assembly includes a reciprocally driven member, the reciprocally driven member operatively connected to a first surface of the flexible barrier. The physical drive assembly reciprocally drives the flexible barrier to a first position in the hollow chamber of the rigid barrier housing during a first position of the physical drive member. During a second position of the physical drive member, the flexible barrier returns to a second position within the hollow chamber of the rigid barrier housing.

The physical drive member is reciprocally driven by a power source. Movement of the flexible barrier between the first position and the second position in the chamber of the rigid barrier housing provides an alternating source of vacuum pressure and the relief of vacuum pressure in a drip tube.

In an embodiment, the drip tube, the rigid barrier housing and the flexible barrier, the mechanical drive assembly, and the power source are located within the perimeter of the reservoir. In another embodiment, the power source and a prime mover are located outside of but adjacent to the perimeter of the reservoir.

### BRIEF DESCRIPTION OF THE DRAWINGS

The invention in its several embodiments will be more fully understood by reference to the following drawings which are for illustrative, and not limiting, purposes only:

FIG. 1 is a schematic cross-sectional assembly view of an embodiment of the invention where the rigid barrier housing, flexible barrier, mechanical drive assembly and power source are located within the perimeter of the reservoir, but are located at a distance from an aperture of the drip tube.

FIG. 2 is a schematic detail view of a portion of the eccentric reciprocal mechanical drive assembly, flexible barrier, and rigid barrier housing of the embodiment of FIG. 1.

FIG. 3 is a schematic illustration of the mechanical drive assembly of the present invention, showing the eccentric drive mechanism, prime mover, controller and power source of the embodiment of FIG. 1.

FIG. 4 is a schematic cross-sectional assembly view of another embodiment of the invention, where the rigid barrier housing, flexible barrier, mechanical drive assembly and power source are located adjacent to and substantially aligned with an aperture at one end of the drip tube.

FIG. 4A is a schematic cross-sectional assembly view of a further embodiment of the embodiment illustrated in FIG. 4, where a portion of the mechanical drive assembly, the power source, the prime mover, and the controller are located outside of and adjacent to the perimeter of the reservoir.

FIG. 5 is a schematic detail assembly view of the eccentric reciprocal mechanical drive assembly, flexible barrier, and rigid barrier housing of the embodiment shown in FIG. 4.

FIG. 6A is a schematic view of another embodiment of the present invention showing a mechanical drive assembly comprising a hollow cylindrical multi-helical grooved rotary-motion-to-reciprocal-motion cam mechanism operatively connected through a follower element to the flexible barrier.

FIG. 6B is a detail view of the pocket in the drive member attached to the flexible barrier, showing the spring-biased pin engaging one of the grooves formed in the cylindrical cam mechanism of the embodiment of FIG. 6A.

FIG. 7 is a detail schematic view of the hollow cylindrical multi-helical grooved rotary-motion-to-reciprocal-motion cam drive system of FIG. 6A, with the gear reduction system, prime mover, controller and power source located at one end of and within the hollow portion of the hollow cylindrical cam mechanism.

FIG. 8 is a detail schematic view of a somewhat more compact version of the relationship between the hollow cylindrically grooved cam mechanism providing a rotary-motion-to-reciprocal-motion drive system and the gear reduction system, compared to the embodiment shown in FIG. 7.

FIG. 9 is a further embodiment of the present invention including a solid rod-like cylindrically multi-grooved rotary-motion-to-reciprocal-motion cam mechanism drive system, with all elements including the rod-like grooved cylinder, gear reduction system, prime mover, controller and power source within the perimeter of the reservoir.

FIG. 10 is a schematic view of a further embodiment of a rotary-motion-to-reciprocal-motion mechanical drive assembly of the present invention, illustrating a multiple-peak-and-dwell rotatable drive element engaging a reciprocally moveable drive shaft, with the reciprocally moveable drive shaft operably connected to a surface of the flexible barrier.

FIG. 11 is a schematic detail view of another embodiment of the device illustrated in FIG. 10, where the physical drive assembly comprises a plurality of multiple-peak-and-dwell rotatable drive elements, where each of the different drive elements, when operatively engaged with the reciprocally moveable drive shaft, provides variable lengths of movement, or throw, of the reciprocally moveable drive shaft.

FIG. 12 is another embodiment of the present invention where a circularly rotating drive gear includes an off-center pin attached to a link that reciprocally moves the flexible barrier between its original position and distended position in or over the rigid barrier housing.

### DETAILED DESCRIPTION OF THE ILLUSTRATED EMBODIMENTS

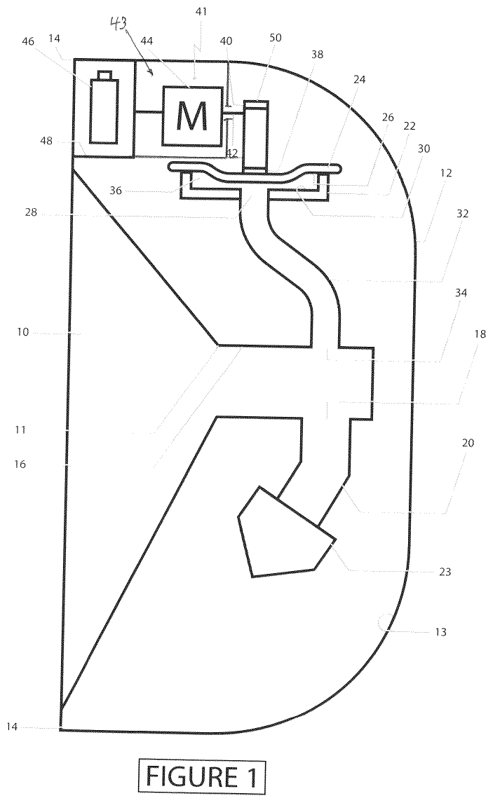

The present embodiment illustrated in FIGS. 1 to 3 is an improved lightweight breast milk collection device including an adaptor 10 configured with a funnel shape to receive a woman's breast in the wide part of the adaptor. A reservoir 12 having a perimeter 13 is attached at 14 rim-to-rim to the adaptor 10. The adaptor 10 has a drip tube 16 extending into reservoir 12 from the narrow end 11 of adaptor 10, the drip tube adapted to receive the nipple of the woman's breast. Aperture 18 is located in drip tube 16, and a tube 20 extends from aperture 18 to a one-way valve 23, which is a duckbill valve in the illustrated embodiment. Any other suitable one-way valve could also be used.

The several embodiments of the present invention dramatically improve the ability of a lactating woman to pump breast milk while in the workplace or other public places or at home without undressing, by providing a compact breast milk collection device that controllably operates to express breast milk while fitting entirely inside the brassiere worn by the woman beyond the view of those around her, while simultaneously providing maximum milk storage capacity in the reservoir.

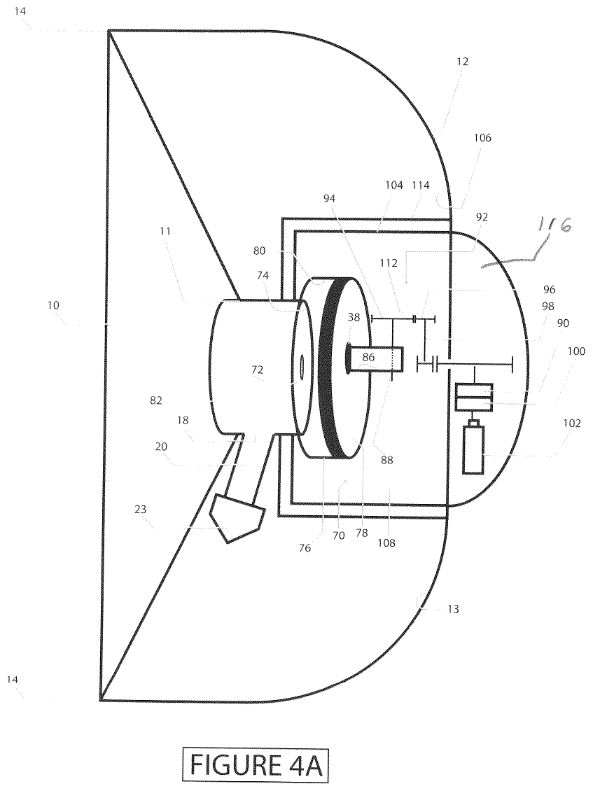

In the embodiment illustrated in FIG. 1, a rigid barrier housing 22 is mounted in the interior of reservoir 12. In alternate embodiments (FIG. 4), rigid barrier housing 22 could be mounted on an external surface of drip tube 16, or at the distal end of drip tube 16. In the illustrated embodiment of FIGS. 1-3, flexible barrier 24 is disposed above rigid barrier housing 22 and extends across the opening 26 formed in the rigid barrier housing. Flexible bladder 24 is made of a flexible rubber or plastic material such that the flexible barrier can expand and contract relative to hollow chamber 36 formed in rigid barrier housing 22, as will be explained.

An aperture 28 is located in the bottom portion 30 of rigid barrier housing 22. A tube or channel 32 communicates with aperture 34 in drip tube 16. The aperture 34 and drip tube 16 are in fluid communication with hollow chamber 36 formed between the underside of flexible barrier 24 and the bottom portion 30 of rigid barrier housing 22. In the illustrated embodiment, a hermetic seal is formed between rigid barrier housing 22 and flexible barrier 24, preventing any overflow milk that may migrate into channel 32 and/or hollow chamber 36 from exiting hollow chamber 36 except through aperture 28.

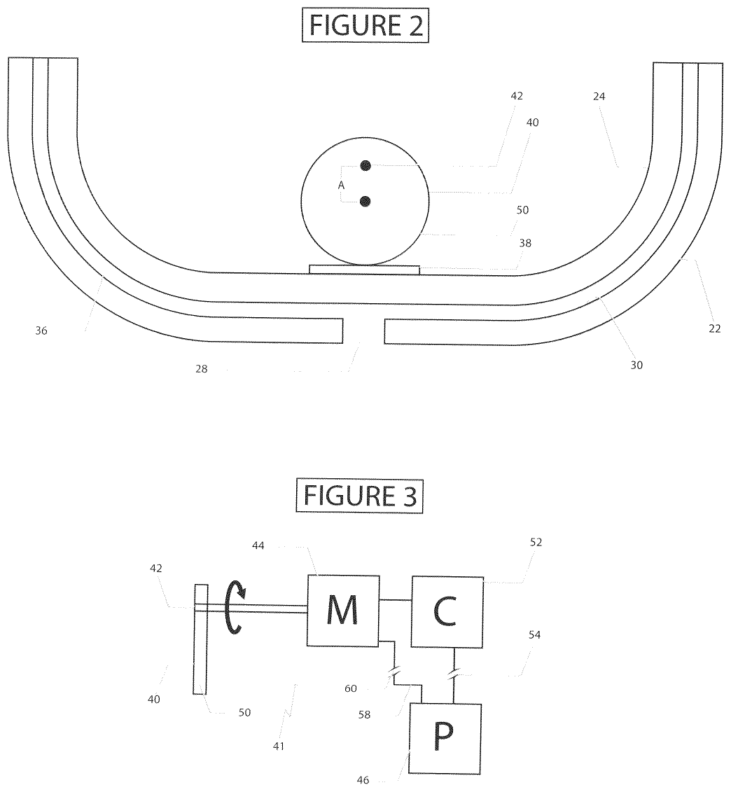

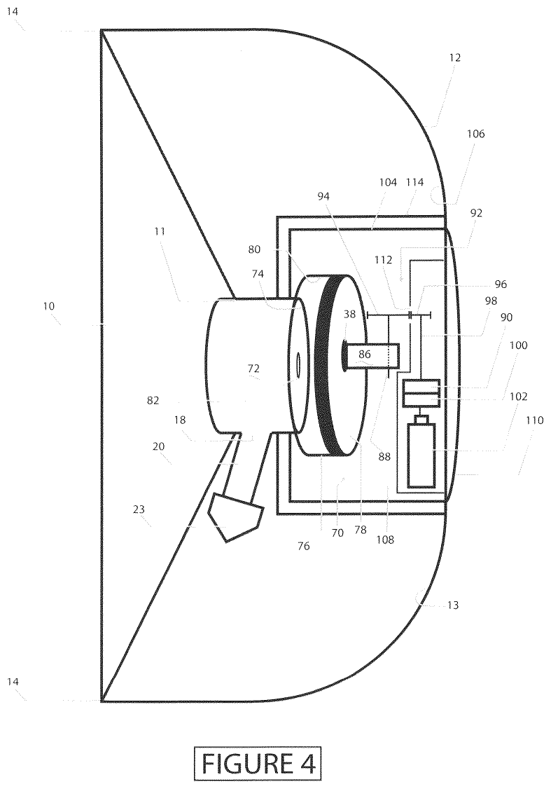

As illustrated in FIGS. 1 and 2, located above and in contact with an upper surface of flexible barrier 24 is a drive assembly 41 (FIGS. 1, 3) comprising a bearing plate or bearing surface 38. The drive assembly 41 also includes a mechanical drive assembly comprising an eccentrically or off-center rotating drive element, such as a drive cam or an off-center mounted drive element 40, that has a circular outer surface 50 in frictional contact with bearing surface 38. Drive shaft 42 connects drive element 40 to a prime mover 44, such as a miniature electric motor as is known in the art. Prime mover 44 may be mounted to an inner surface of the reservoir 12 or a surface of adaptor 10, and prime mover 44 is powered by an electric power source 46, such as a rechargeable battery. Upon rotation of drive element 40 by prime mover 44, flexible barrier 24 moves reciprocally in hollow chamber 36, providing a source of alternating vacuum force and relief of vacuum force in chamber 36 of rigid barrier housing 22. The alternating application and relief of vacuum force is applied through channel 32 to drip tube 16, and to the nipple located in drip tube 16, for the expression of milk from the woman's breast. As described above, the drive assembly 41 comprises a rotating-motion-to-reciprocal-motion mechanical drive assembly extending between the power source 46 and the upper surface of flexible barrier 24.

Once flexible barrier 24 moves away from its distended position (FIGS. 1, 2) over rigid barrier housing 22 to its original position by a first partial cycle of rotation of drive element 40, the inherent flexible nature of flexible barrier 24 will provide the internal force necessary to return the flexible barrier to its original or first position as drive element 40 rotates through a second partial cycle.

In the illustrated embodiment of FIG. 1, rigid barrier housing 22 is suitably attached to an inner surface of reservoir 12, or in alternate embodiments, to an outer surface or an end surface of drip tube 16. Drive member 40 and prime mover 44 are also suitably attached to one of the solid or fixed elements of the breast milk collection device, such that drive member 40 and prime mover 44 are fully isolated in compartment 43 from the breast milk in reservoir 12 by the use of appropriate seals (not shown), or from overflow milk that may have migrated into channel 32 and/or into hollow chamber 36. Further, power source 46 may be located outside of the outer surface of reservoir 12 in a compartment 48 for ease of replacement and/or recharging. The recharging electric power connection may be a wired connection, or power may be transferred by induction or other wireless power transfer systems (WPT or WET) as are known in the art.

FIG. 2 is a schematic detail diagram of the rigid barrier housing 22, the flexible barrier 24 and the eccentric or reciprocating drive element 40 shown in FIG. 1. In FIG. 2, drive element 40 is shown in a side elevation view with a circular outer surface 50, and the drive element is mounted for rotation with shaft 42 at an offset distance “A” from the center of drive element 40. Upon rotation of shaft 42 and drive element 40, the portion of outer surface 50 in contact with bearing surface 38 will have a vertical component, causing flexible bladder 24 to move reciprocally up and down, causing a vacuum force to be alternately applied and relieved in hollow chamber 36 as described above.

FIG. 3 is a schematic illustration of the connection between eccentric drive element 40 and power source 46 of the embodiment of FIG. 1. Power source 46 can be any one of known small power sources, such as a rechargeable DC battery, a suitable stack of circular batteries, or the like. Power source 46 can be directly wired to controller 52 to vary the speed of motor 44 through controller 52 as determined by the user. Also, the power source 46 can be coupled to controller 52 by a near-field wireless power transfer (WPT or WET) or inductive system 54 as is known in the art. In the illustrated embodiment of FIG. 3, power source 46 is also electrically connected to prime mover 44 through wire 58, or through near-field wireless power transfer (WPT or WET) system 60. As stated above, use of a WPT or WET system allows power source 46 to be located in a compartment 48 (FIG. 1) outside of perimeter 13 of reservoir 12, while prime mover 44, rigid barrier housing 22 and flexible bladder 24 are located inside perimeter 13 of reservoir 12. In the embodiment of FIG. 1, power source 46 is a rechargeable battery located in separate compartment 48 inside perimeter 13 of reservoir 12.

In another embodiment, not shown, a plurality of drive elements 40 may be attached for rotation by drive shaft 42. In this embodiment, each drive element comprises a removable and replaceable off-center rotating cam element, where each off-center rotating cam element has a different radial dimension compared to the other off-center rotating cams.

In operation, the breast milk collection device illustrated in FIGS. 1-3 is assembled, after cleaning, substantially as shown schematically in FIG. 1. Adaptor 10 and reservoir 12 are detachably joined at rim-to-rim attachment 14 to form an integral one-piece breast milk collection device. One or a pair of breast milk collection devices are then placed over the user's breasts and into one or each of the cups of the nursing or standard brassiere worn by the lactating woman. The breast milk collection devices are held in place against the breast and fully concealed by, for example, the front upper rim of the brassiere. Each breast will firmly fit into a respective funnel-shaped portion of the adaptor 10, with the nipple(s) extending forwardly into drip tube 16. The contact between the breast and the adaptor 10 creates a seal, such that the vacuum forces applied to the outer end of the breast do not escape between the breast and the adaptor 10.

Prime mover, or motor, 44 is then actuated, after control parameters in controller 52 are set by the user. Prime mover 44 rotates drive shaft 42 in a clockwise direction (FIG. 2). As shaft 42 rotates, drive element 40 also rotates along with shaft 42. Since shaft 42 is located off center with respect to the center of drive element 40 (“A”), drive element 40 is eccentrically rotated. As drive element 40 rotates, the outer surface 50 of the drive element contacts bearing surface 38 on the upper surface of flexible barrier 24, initially moving flexible bladder 24 from its original position to a second distended position (FIG. 2) in a direction toward the bottom portion 30 of rigid barrier housing 22. As drive member 40 continues to rotate, the distance between drive shaft 42 and outer surface 50 of drive member 40 in contact with bearing plate 38 decreases, and flexible barrier 24 moves upward to its original or first position while at all times covering the entirety of opening 26 of rigid barrier housing 22.

As flexible barrier 24 moves from the distended position shown in FIGS. 1 and 2 to its original position, vacuum force is created in hollow chamber 36. This vacuum force is transmitted through aperture 28, tube 32 and aperture 34 to the user's nipple extending laterally into drip tube 16, causing milk to be expressed from the breast into drip tube 16. Upon movement of flexible bladder 24 to its second or distended position (FIGS. 1, 2), the vacuum force in hollow chamber 36 is relieved, and the increase of pressure in drip tube 16 forces the milk in the drip tube to pass through aperture 18, tube 20, and through one way valve 23 into reservoir 12. As drive element 40 continues its eccentric rotation, the above-described cycle repeats until the milk flow from the breast ceases and the user turns prime mover 44 to an off position. Suitable fluid seals (not shown) are used to prevent the milk as it rises in reservoir 12 from contacting the mechanical and electrical features of the presently disclosed breast milk collection device.

FIG. 4 illustrates a further embodiment of the presently disclosed breast milk collection device. As in the embodiment of FIG. 1, an adaptor 10 is configured with a funnel shape to receive a lactating woman's breast in the wide part of the adaptor 10. A milk-receiving reservoir 12 having a perimeter 13 is removably attached at 14 rim-to-rim to adaptor 10. A drip tube 72 extends into reservoir 12 from the narrow end 11 of adaptor 10, and the drip tube 72 is adapted to receive the nipple of the lactating woman's breast when the breast is inserted into adaptor 10. Aperture 18 is located in drip tube 72, and tube 20 extends from aperture 18 into reservoir 12 through a one-way valve 23, which is a duckbill valve in the embodiment illustrated in FIG. 4. Any other suitable one-way valve as is known in the art could also be used in this embodiment.

In the embodiment illustrated in FIG. 4, the source 70 of application and relief of vacuum force in the drip tube 72 is physically located at the distal end 74 of drip tube 72. A rigid barrier housing 76 extends outward from distal end 74 of drip tube 72, and a flexible barrier 78 extends across the entirety of the opening of rigid barrier housing 76. Flexible barrier 78 comprises a flexible rubber or plastic material that allows the flexible barrier 78 to expand and contract in hollow chamber 80 formed in rigid barrier housing 76, as will be explained. Hollow chamber 80 is formed between one side of flexible barrier 78 and the distal end 74 of drip tube 72.

As explained previously with regard to flexible barrier 24 (FIGS. 1, 2), the inherent flexible nature of flexible barrier 78 in the embodiment of FIG. 4 provides the internal force necessary to return the flexible barrier to its original position after the flexible barrier has been moved away from its original position to a distended position, as will be explained.

In the embodiment of FIG. 4, an aperture 82 is located at the bottom of hollow chamber 80 to allow fluid communication between hollow chamber 80 and the interior of drip tube 72. In an alternative embodiment, aperture 82 is eliminated, and the entire volume of hollow chamber 80 communicates with drip tube 72. Aperture 82 and drip tube 72 are also in fluid communication with hollow chamber 80 formed between one side of flexible barrier 78 and rigid barrier housing 76. In the illustrated embodiment of FIG. 4, a hermetic seal is formed between rigid barrier housing 76 and flexible barrier 78, preventing any overflow milk that may attempt to migrate from drip tube 72 to hollow chamber 80 from exiting hollow chamber 80.

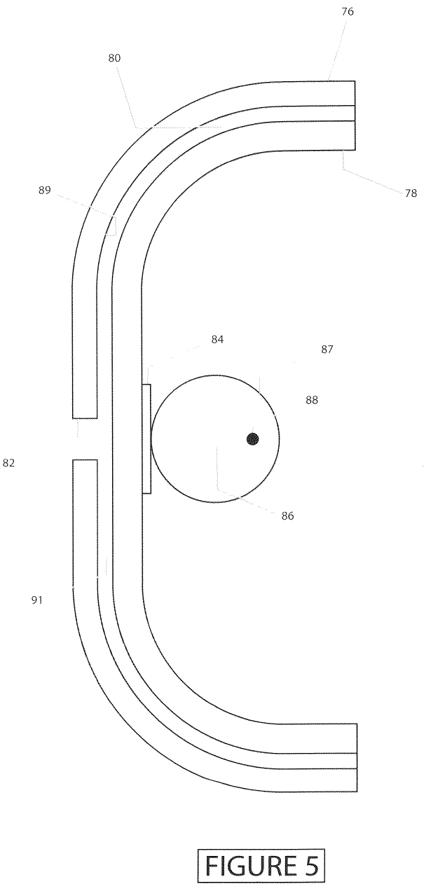

Located on the side of flexible barrier 78 opposite the side of the flexible barrier that faces drip tube 72 in the illustrated embodiment of FIGS. 4 and 5, is a bearing plate or bearing surface 84 (FIG. 5). An eccentrically or off-center rotating drive element, such as a drive cam or off-center mounted drive element 86 is in frictional contact with bearing surface 84. Rotating drive shaft 88 operatively connects drive element 86 to a prime mover 90 (FIG. 4) such as a miniature electric motor as is known in the art, for example miniature electric motors used currently to drive electric toothbrushes. For example, such miniature electric motors used in toothbrushes are typically in the range of 24 mm in diameter and 30 mm in length or less.

As illustrated in FIG. 4, drive shaft 88 is operatively connected to prime mover 90 through a gear reduction system 92 comprising, in the illustrated embodiment of FIG. 4, a drive gear 94 firmly attached to rotating drive shaft 88. The teeth of smaller pinion gear 96 engage the teeth of drive gear 94, and pinion gear 96 is firmly attached to drive shaft 98. Drive shaft 98 is rotated upon actuation of prime mover 90.

Referring to FIG. 4, the rotation of drive shaft 98 by prime mover 90 causes shaft 88 to rotate due to the interaction between pinion gear 96 and drive gear 94. The rotation of shaft 88 causes off center drive element 86 to rotate, as will be explained. In the illustrated embodiment of FIGS. 4 and 5, the ratio of the diameters of drive gear 94 and pinion gear 96 is typically established so that drive shaft 88 rotates at approximately forty five rotations per minute. As explained below, this speed of rotation of drive shaft 88 may be modified as a user desires.

Electronically connected to prime mover 90 is a control system 100 designed to be operated by the user of the illustrated breast pump to achieve higher or lower rates of breast milk pumping. A power source for both prime mover 90 and control system 100 is provided by rechargeable or removable battery 102.

A removable generally U-shaped housing 104 extends from the distal end 74 of drip tube 72 to the inside surface 106 of reservoir 12. Housing 104 provides a hermetic seal around rigid barrier housing 76, flexible barrier 78, drive element 86, drive gear 94, pinion gear 96, prime mover 90, control system 100, and battery 102. This hermetic seal prevents milk in reservoir 12 and in chamber 80 from contacting any of the power or mechanical systems of the breast pump as milk rises in reservoir 12 vertically beyond drip tube 72. The removability of housing 104 allows flexible barrier 78 to be removed from the breast pump for cleaning. As seen in the embodiment of FIG. 4, hollow drip tube 72, flexible barrier 78 and rigid barrier housing 76 are in axial alignment.

Another housing 108 is removably embedded in housing 104, and prime mover 90, pinion gear 96, control system 100 and battery 102 are lodged in housing 108. Housing 108 includes an outer facing plate 110 that substantially conforms to the outer shape of reservoir 12 as shown in FIG. 4. Housing 108 is also removably installed in housing 104, so that housing 108 can be removed to change or recharge battery 102 if required. Adjacent to pinon gear 96 is an aperture 112 through which pinion gear 96 partially extends when housing 108 is fully installed in housing 104. This allows pinion gear 96 to operably engage drive gear 94 when housing 108 is installed in housing 104. In the embodiment illustrated in FIG. 4, housing 108 is removably held in place in housing 104 by a snap-fit connection between housing 108, outer facing plate 110 and housing 104, or by any other suitable detachable connection as is known in the art.

In another embodiment, battery 102 can be coupled to a near-field wireless power transfer (WPT or WET) or inductive system (not shown) as is known in the art. The use of a WPT, WET or inductive system would allow battery 102 to be recharged or kept charged without being removed from housing 108.

A breast milk collection device constructed in accordance with the above description of the first and second embodiments of FIGS. 1-5 would provide that all of the power and structural elements could be located inside perimeter 13 of reservoir 12, thus eliminating the need for a vacuum pump and pump housing to extend above and/or outside of the brassiere worn by the user, as is common in several breast pump devices marketed and sold today. Also, the presently disclosed breast pump embodiments can have a larger reservoir while allowing the entire breast pump to fit discreetly and unseen in the brassiere of the lactating mother while collecting breast milk.

In the operation of the embodiment of the present invention shown in FIGS. 4 and 5, battery 102 is checked to be sure the battery is fully charged. Housing 108 with prime mover 90 and control system 100, is then slidably installed into housing 104 until a snap-fit connection is made and the teeth of pinion gear 96 mesh with the teeth of drive gear 94 through aperture 112. The control system 100 includes several control buttons (not shown) on the outer surface of outer facing plate 110, so the control buttons are readily accessible to the user. The buttons are used to select the operation parameters embedded in control system 100, such as the speed of the rotation of prime mover 90, on and off control, and the like.

The lactating mother places her breast in funnel shaped adaptor 10 with the nipple extending into drip tube 72. The entire breast milk collection device is simultaneously located inside a cup of the user's nursing or regular brassiere. Adaptor 10 is manipulated into position over the breast so that a hermetic seal is formed between the breast and adaptor 10.

After having checked that battery 102 is sufficiently charged, prime mover 90 is actuated by manual manipulation of control system 100, which also activates prime mover 90 causing rotation of the drive shaft 98. Gear reduction system 92 rotates drive shaft 88 at a rate of approximately forty five rpm, and off center drive element 86 is rotated at the same rate. As off center drive element 86 rotates, the outer surface 87 (FIG. 5) of drive element 86 engages bearing plate or surface 84 (FIG. 5), moving flexible barrier 78 from a first or original position to a second distended position, the second distended position being in a direction towards bottom surface 89 of rigid barrier housing 76, as seen in FIG. 5. As off center drive element 86 continues to rotate, flexible barrier 78 moves away from bottom surface 89 of rigid barrier housing 76 and back to the first or original position across hollow chamber 80 of rigid barrier housing 76. As explained previously, the inherent flexibility of flexible barrier 78 allows the flexible barrier to return to its first or original position after the flexible barrier 78 is “stretched” to its second or distended position adjacent bottom surface 89 of rigid barrier housing 76.

As seen in FIGS. 4 and 5, an aperture 82 is formed in bottom surface 89 of rigid barrier housing 76, such that the portion of hollow chamber 91 (FIG. 5) formed on one side of flexible barrier 78 communicates with drip tube 72, and also communicates with the user's nipple lodged in drip tube 72. As seen in FIGS. 4 and 5, when flexible barrier 78 moves from its second or distended position to its original or first position, a vacuum force is created in hollow chamber portion 91. This vacuum force is communicated via aperture 82 to drip tube 72 and to the nipple of the user, where the vacuum force on the nipple results in the expression of milk from the user's breast into drip tube 72.

As off center drive element 86 continues to rotate, flexible barrier 78 moves to its second or distended position, resulting in the relief of vacuum pressure in drip tube 72, and increasing the pressure in drip tube 72 above vacuum pressure. This increased pressure drives the milk in drip tube 72 through aperture 18 and through tube or channel 20 and one way valve 23 into the internal volume of reservoir 12. This action repeats until the flow of milk from the breast ceases, and prime mover 90 is turned off. At this point, the adaptor 10 is withdrawn from the user's breast, and the parts are cleaned in preparation for subsequent use.

As seen in the illustrated embodiment of FIG. 4, rigid barrier housing 76, flexible barrier 78, gear reduction system 92 and housing 108 are located inside removable housing 104. Removable housing 104 is slidably inserted and removed from chamber 114 (FIG. 4) in reservoir 12. Suitable latching devices (not shown) are located on outer facing plate 110 to releasably insert housing 104 in chamber 114. In this manner, flexible barrier 78 can be removed for cleaning after each use, and reinstalled for further use. Also, the removal of flexible barrier 78 from rigid barrier housing 76 allows hollow chamber 80 of rigid barrier housing 76 to be cleaned after each use. After each use, reservoir 12 is detached from adaptor 10, which permits ease of cleaning of reservoir 12, adaptor 10, one-way valve 23, and drip tube 72.

Housing 108 is also removably installed in housing 104 to allow access to prime mover 90, control system 100, and battery or power source 102, if necessary. For example, if power source 102 is not a rechargeable battery, power source 102 may need to be replaced. In the illustrated embodiment, as explained previously, power source 102 is a rechargeable battery.

It is contemplated within the scope of the present invention that rigid barrier housing 22, flexible barrier 24, and drive element 86 may be located in reservoir 12, and that the configuration of the several elements of the presently disclosed wearable breast milk collection system may be rearranged to accommodate engineering, manufacturing and aesthetic considerations.

FIG. 4A illustrates a further embodiment of the presently disclosed breast milk collection device of FIG. 4 where a portion of the mechanical drive assembly 92, the power source 102, the prime mover 90 and the controller 100 are located outside of and adjacent to the perimeter 13 of reservoir 12. As seen in FIG. 4A, a compartment 116 is formed outside of and adjacent to reservoir 12. By the use of suitable fluid seals (not shown), the internal volume of compartment 116 is isolated from the breast milk collected in the internal volume of reservoir 12. As a result, the portion of drive assembly 92 in compartment 116, and the power source 102, the prime mover 90 and the controller 100 are protected against coming into contact with the breast milk in reservoir 12. In all other respects, the structure and operation of the breast milk collection device embodiment of FIG. 4A is the same as the embodiment of FIG. 4. Like numbers in FIGS. 4 and 4A identify like elements.

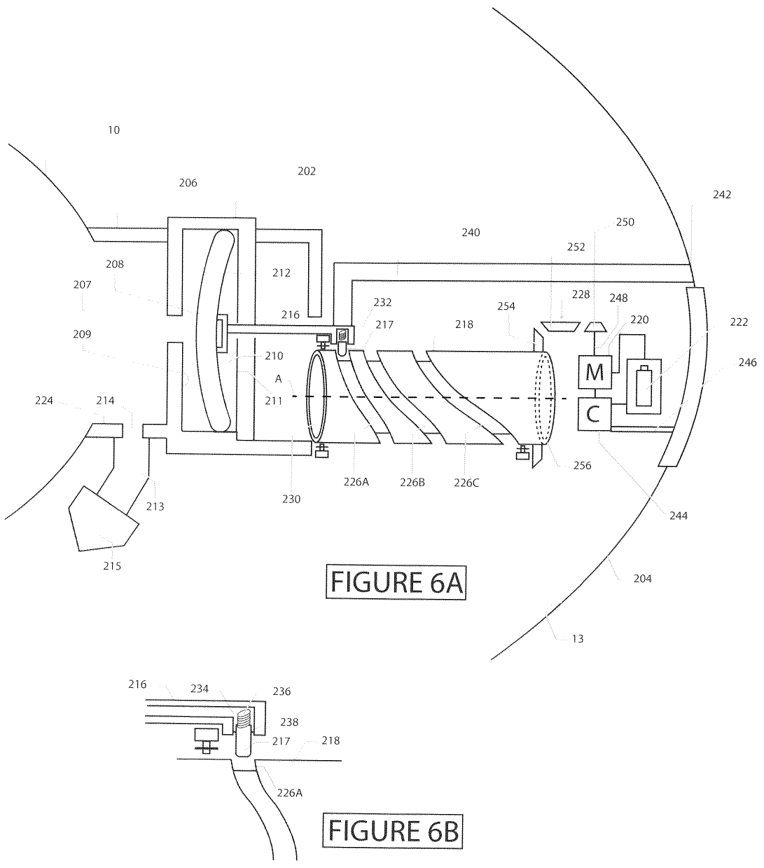

In an additional embodiment of the present invention illustrated in FIGS. 6A and 6B, a rigid barrier housing 202 is mounted at the distal end of drip tube 206 in the interior of reservoir 204. An aperture 207 is located in surface 209 of rigid barrier housing 202. A flexible barrier 208, as described previously, is disposed in the rigid barrier housing 202 at the distal end of drip tube 206, and the flexible barrier 208 extends across an opening 210 formed in the rigid barrier housing 202. The flexible barrier 208 is made of a flexible rubber or plastic material such that the flexible barrier can expand and contract relative to hollow chamber 212 formed in rigid barrier housing 202, as explained in previous embodiments. An aperture 214 is located at the bottom portion of drip tube 206. The aperture 214 of drip tube 206 is in fluid communication with hollow tube 213, one-way valve 215, and reservoir 12.

Located in contact with an upper surface 211 of flexible barrier 208 in the embodiment of FIGS. 6A and 6B is a horizontally moveable drive shaft 216 as viewed in FIG. 6A. A rotating drive element, such as hollow rotatable grooved cylindrical cam mechanism 218 is in frictional contact with an engagement element such as spring-actuated pin 217 that acts as a cam riding in a selected groove 226A, B, C of rotating cylindrical cam member 218. The horizontally moveable drive shaft 216 operably connects the cylindrical cam element 218 to drive shaft 216 to provide reciprocal linear movement of drive shaft 216. Cylindrical cam element 218 is operatively connected to a prime mover 220, such as a miniature electric motor as is known in the art, through gear reduction system 228. Controller 244 is manually operable to vary the rate of rotation of prime mover 220. Prime mover 220 is powered by an electric power source 222, such as a rechargeable battery or the like.

In the embodiment illustrated in FIGS. 6A and 6B, cylindrical cam mechanism 218 is hollow, as shown at 230. The outer surface of cylindrical cam mechanism 218 includes a plurality of grooves 226A, 226B and 226C, which grooves simultaneously extend around the circumference and extend along the axis of cylindrical cam mechanism 218. In the illustrated embodiment, three grooves 226A, B and C are shown, however more or less grooves may be suitable to vary the desired extent of the movement of flexible bladder 208 in hollow chamber 212 by the reciprocal linear movement of drive shaft 216, as will be explained.

The outer end 232 of horizontally moveable drive shaft 216 includes a pocket 234, as seen in FIG. 6B. Pin 217 is movably mounted in pocket 234, and spring 236 biases pin 217 downward, as shown in FIG. 6B. A flange assembly 238 maintains pin 217 in pocket 234 against the bias of spring 236.

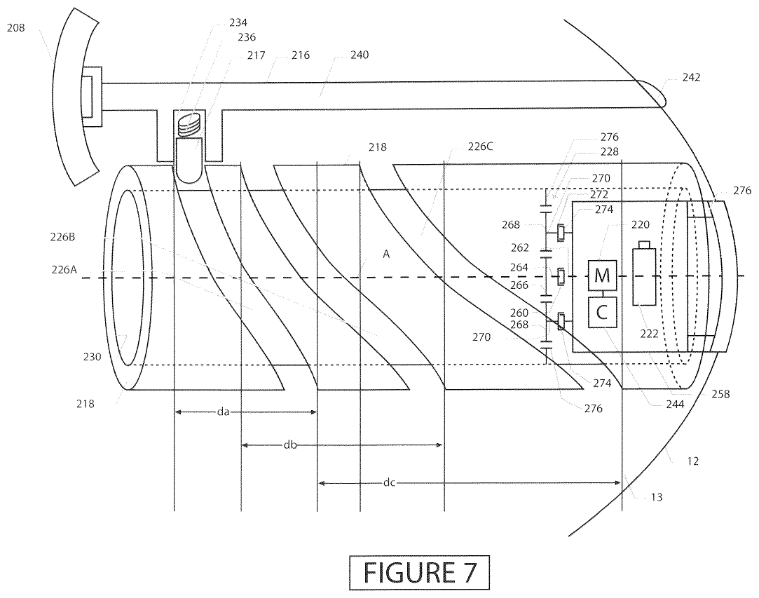

As illustrated in the embodiments of FIGS. 6A, 6B and 7-9, horizontally moveable drive shaft 216 extends above the outer surface of cylindrical cam mechanism 218, with pin 217 biased by spring 236 to extend into one of grooves 226A, B or C. In each of FIGS. 6, 6A, 6B and 7-9, pin 217 is shown extending into groove 226A, by way of example. Outer end 240 of drive shaft 216 extends horizontally, in the embodiment illustrated in FIGS. 6, 7 to the perimeter 13 of reservoir 204, where a manually operable control device 242 allows the user to position and reposition the horizontal location of pocket 234 and pin 217 relative to one of grooves 226A, 226B or 226C of cylindrical cam mechanism 218. As drive shaft 216 moves horizontally, the curved bottom end of pin 217 engages the vertical side of the groove in which the pin 217 initially extends, and pin 217 moves upward until the curved bottom end of pin 217 engages the outer surface of cylindrical cam mechanism 218. Upon further horizontal movement of drive shaft 216 by the user, pin 217 subsequently is biased into the next adjacent groove 226. In the embodiment of FIG. 6A for example, the next adjacent groove 226 is groove 226B.

Repeating the above-described repositioning of horizontally moveable drive shaft 216 will result in pin 217 engaging either groove 226C or 226A. As will be explained, the distance drive shaft 216 travels, and the amount of movement of flexible barrier 208 in hollow chamber 212 is a function of which groove 226A, B or C pin 217 extends into upon rotation of cylindrical cam mechanism 218, as will be explained.

As illustrated in FIG. 6A, prime mover 220 is operatively connected to a controller 244, which controller is manually operable through control link 246. Rechargeable battery 222 is electrically connected to both prime mover 220 and to controller 244. Shaft 248 extends from and is rotated by actuation of prime mover 220. In the embodiment of FIG. 6A, gear reduction system 228 comprises a beveled small pinion gear 250 that is rotated by shaft 248, and pinion gear 250 meshes with relatively larger beveled gear 252. A further beveled gear structure 254 is fixedly mounted on the outer surface of cylindrical cam mechanism 218 in the illustrated embodiment. As seen in FIG. 6A, rotation of shaft 248 by prime mover 220 results in the rotation of cylindrical cam mechanism 218 in the illustrated embodiment in a circular motion around central axis A. Gears 250, 252 and 254 provide the gear reduction system 228 in which cylindrical cam mechanism 218 rotates at approximately forty five rotations per minute in the illustrated embodiment. If desired, other gear reduction systems as are known in the art can be used to provide the desired rate of rotation of cylindrical cam mechanism 218.

In the embodiment of FIG. 6A, prime mover 220, gear reduction system 228, controller 244 and battery 222 are located inside of reservoir 12, but outside of the end 256 of cylindrical cam mechanism 218. FIG. 7 illustrates a further embodiment of the present invention where prime mover 220, controller 244 and rechargeable battery 222 are located in a compartment 258. The compartment 258 with prime mover 220, controller 244 and battery 222 is removably installed in reservoir 12 as shown in FIG. 7. When compartment 258 is installed in reservoir 12, a self-operating releasable snap connection 260 connects shaft 262 of prime mover 220 to shaft 264, and shaft 264 is fixedly attached to relatively small pinion gear 266 of gear reduction system 228. Pinion gear 266 meshes with relatively larger spur gears 268, and each spur gear 268 rotates about a corresponding fixed shaft 270. Each shaft 270 is removably attached by a snap connection 272 to a shaft 274. Shaft 274 is rigidly connected to removable compartment 258 such that each shaft 270 remains stationary when compartment 258 is installed in reservoir 12 as shown in FIG. 7. Located on the interior surface of cylindrical cam mechanism 218 and aligned with spur gears 268 is a circular ring gear structure 276. As seen in FIG. 7, spur gears 268 each mesh with ring gear structure 276.

Referring to the embodiment of FIG. 7, when compartment 258 is installed in reservoir 12 and snap connections 260 and 272 engage their respective shafts 264 and 270, activation of prime mover 220 causes spur gear 266 to rotate about shaft 264, and spur gears 268 to rotate about shafts 270. Spur gears 268 also mesh with gear structure 276 integral with an internal surface of cylindrical cam mechanism 218, resulting in the rotation of cylindrical cam mechanism 218 about central axis A. As in the embodiment of FIG. 6A, gear reduction system 228 is configured such that cylindrical cam mechanism 218 rotates about axis A at approximately forty five rotations per minute.

Referring to the embodiments of FIGS. 6A and 7, grooves 226A, B and C provide several pathways for pin 217 to move reciprocally parallel to axis A, which pathways are each configured differently as the grooves 226A, B and C extend around the outer surface of cylindrical cam mechanism 218 in varied configurations. As illustrated in FIG. 7, groove 226A provides a pathway for pin 217 that extends linearly horizontally a maximum distance of da in a direction parallel to axis A. Groove 226B provides a pathway for pin 217 somewhat adjacent to groove 226A. However, groove 226B is configured to provide a pathway that extends linearly horizontally a maximum distance of db in a direction parallel to axis A. In the illustrated embodiment of FIG. 7, distance db is greater than distance da.

As also shown in FIG. 7, groove 226C provides a pathway for pin 217 somewhat adjacent to groove 226B. However, groove 226C extends linearly horizontally a maximum distance of dc in a direction parallel to axis A. In the illustrated embodiment of FIG. 7, distance dc is greater than distance da and distance db. By way of example, distance da could be ¼ inch, distance db could be ⅓ inch, and distance dc could be ½ inch. These distances will vary in accordance with engineering considerations dictating the desired amount of horizontal movement of flexible barrier 208, as seen in FIG. 7.

In operation of the embodiments of FIGS. 6A, 6B, and 7, drive shaft 216 is moved horizontally by the user's manipulation of manual control device 242, resulting in spring loaded pin 217 extending into either groove 226A, 226B or 226C. For purposes of illustration, pin 217 extends into groove 226A. After the user's breast is inserted into adaptor 10 forming a seal between the breast and the adaptor, the user then activates prime mover 220, resulting in the rotation of cylindrical cam mechanism 218 about axis A via gear reduction system 228. As cylindrical cam mechanism 218 rotates, pin 217 moves along the pathway defined by groove 226A, providing reciprocal linear horizontal movement to pin 217 and to drive shaft 216 a distance defined by da. It is understood by those skilled in the art that the rotation of cylindrical cam mechanism 218 results in the reciprocal linear movement of drive shaft 216. This combination of elements provides one of a variety of rotary-motion-to-reciprocal-linear-motion mechanisms. In the illustrated embodiment, the horizontal movement of drive shaft 216 is equal to da (FIG. 7). Since drive shaft 216 is attached to flexible barrier 208, the flexible barrier 208 will also move in chamber 212 a distance da from its distended position (FIG. 6) to its original position creating a vacuum force in drip tube 206, resulting in the expression of milk from the user's breast through the nipple that extends in drip tube 206. As cylindrical cam mechanism 218 continues to rotate, at about one hundred eighty degrees of rotation from start, pin 217 and drive shaft 216 will reverse horizontal direction as pin 217 continues to move in the pathway defined by groove 226A. This movement causes flexible barrier 208 to move from its original position to its distended position (FIG. 6A), thus relieving the vacuum force in drip tube 206, providing sufficient force to convey breast milk through aperture 214 of drip tube 206, through one-way valve 215 and into reservoir 12. Once pin 217 reaches the position in groove 226A shown in FIGS. 6 and 7, flexible barrier 208 is moved to its distended position (distance da), and the cycle of operation described above is repeated, cyclically expressing milk from the user's breast in a first one hundred eighty degree cycle of rotation of cylindrical cam mechanism 218, and driving milk into reservoir 12 during the second one hundred eighty degree cycle of rotation of cylindrical cam mechanism 218.

As mentioned previously, there may be situations where it is desirable from the user's standpoint to vary the amount of vacuum force applied to the nipple in drip tube 206, for example, to increase the rate or volume of flow of milk from the user's breast. This can be accomplished in the embodiments shown in FIGS. 6A, 6B and 7 by horizontally manipulating the outer end 240 of drive shaft 216 such that pin 217 is moved from groove 226A to either groove 226B or groove 226C. If pin 217 is lodged in groove 226B, drive shaft 216 will move linearly horizontally a distance of db (FIG. 7), where distance db is greater than distance da. As a result, the distance db flexible barrier 208 moves between its original position and its distended position during rotation of cylindrical cam mechanism 218 is increased, thus increasing the vacuum force applied to the nipple in drip tube 206, resulting in increased milk flow into the reservoir.

In the illustrated embodiments of FIGS. 6 and 7, the vacuum force applied to the nipple in drip tube 206 can be further increased, if desired, by moving pin 217 to engage groove 226C of cylindrical cam mechanism 218, as described above. Since the distance drive shaft 216 and flexible barrier 208 will move is equal to distance dc, the vacuum force applied to the nipple is correspondingly increased.

In the embodiment of FIG. 6A, the gear reduction system 228, prime mover 220, controller 244 and power source 222 are outside of the end 256 of hollow cylindrical cam mechanism 218. In the embodiment illustrated in FIG. 7, the gear reduction system 228, prime mover 220, controller 244 and power source 222 are located inside the hollow portion of cylindrical cam mechanism 218 adjacent the inner perimeter 13 of reservoir 12. This configuration increases the milk collection volume of reservoir 12.

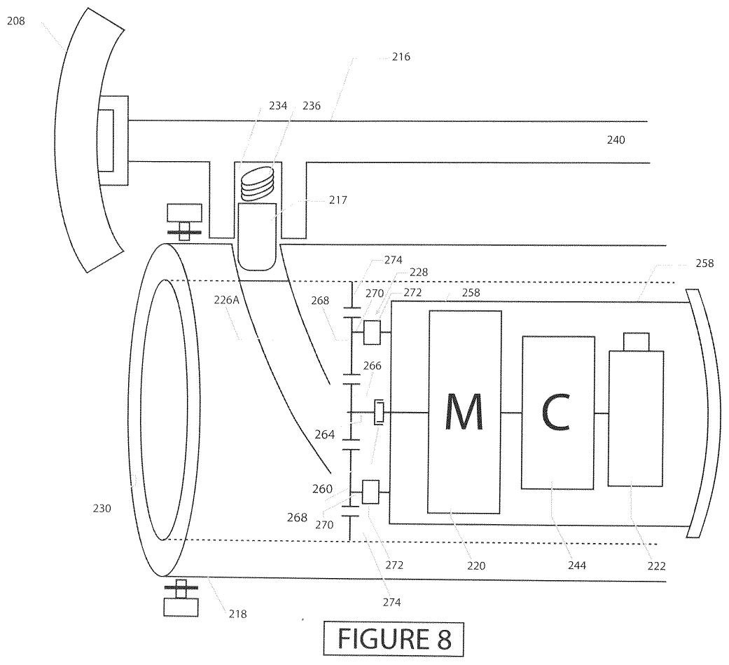

FIG. 8 illustrates an embodiment of the present invention similar to the embodiment of FIG. 7, except that the prime mover 220, controller 244, power source 222 and gear reduction system 228 are located inside of and adjacent the left end of hollow cylindrical cam mechanism 218. In all other respects, the structure and operation of the rotary-motion-to-reciprocal-motion system(s) of the embodiment of FIG. 8 are the same as in the embodiment illustrated in FIG. 7.

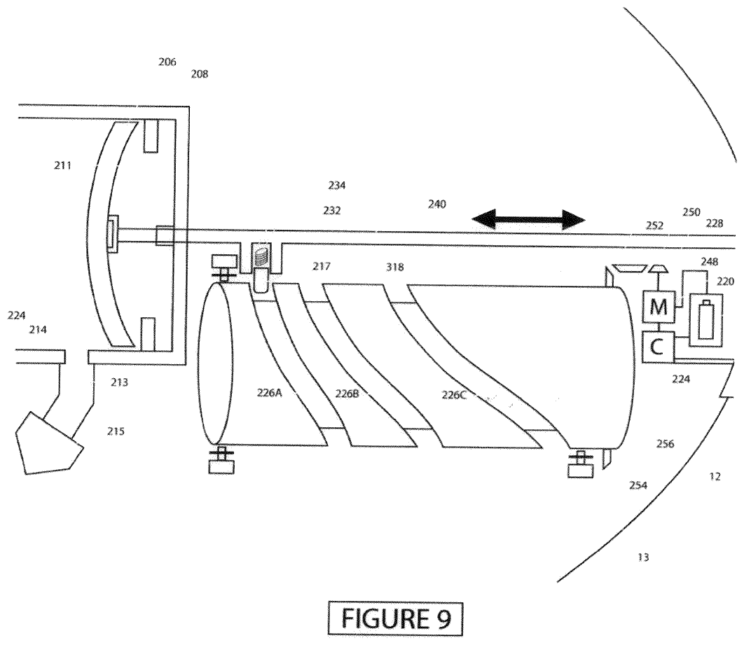

FIG. 9 illustrates an embodiment of the structural features of the breast milk collection device shown in FIG. 6A, except the hollow cylindrical cam mechanism 218 is replaced with a solid bar-like cylindrical cam mechanism 318 with grooves 226A, 226B and 226C extending circularly and axially on the outer surface of solid cylindrical cam mechanism 318. Like elements in FIG. 6A and FIG. 9 identify similar elements in each figure. In all respects, other than the use of a solid cylindrical cam mechanism 318 in place of the hollow cylindrical cam mechanism 218 of FIG. 6, the structure and operation of the embodiment of FIG. 9 is the same as the structure and operation of the embodiment of FIG. 6A.

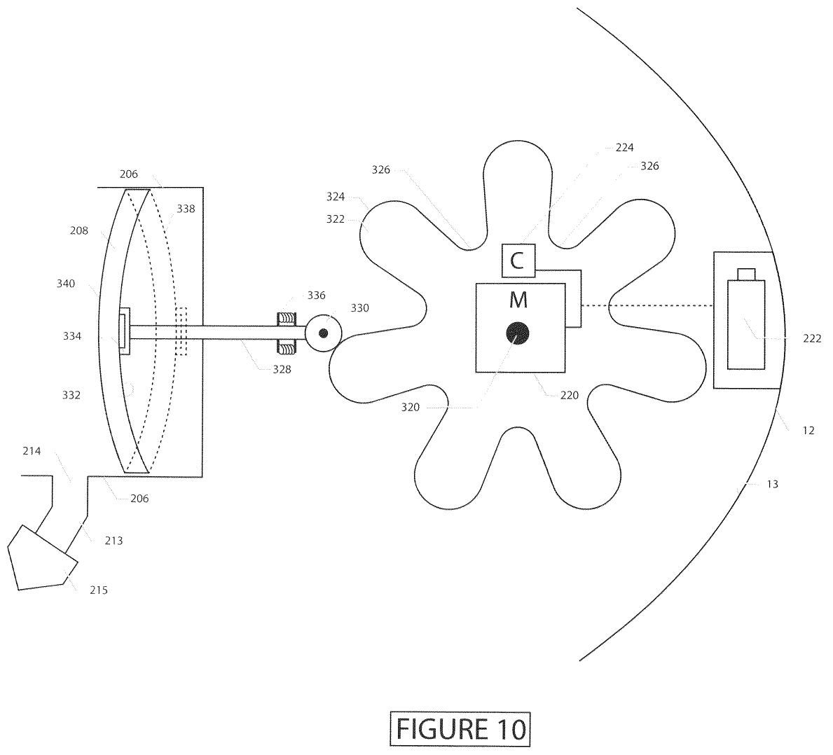

FIG. 10 schematically illustrates a further embodiment of a rotary-motion-to-reciprocal-motion drive mechanism located inside perimeter 13 of reservoir 12 that could be used to provide reciprocal motion to flexible barrier 208 upon the rotation of prime mover 220. In the embodiment illustrated in FIG. 10, prime mover 220, when activated by controller 224, rotates shaft 320. Fixedly attached to shaft 320 is a somewhat flower petal-shaped cam mechanism 322 having a plurality of peak surface points 324 and dwell surface points 326. Drive shaft 328 mounts a rotatable cam follower wheel 330 at one end of the drive shaft, and the other end of drive shaft 328 is attached to surface 332 of flexible barrier 208 as shown at 334. Drive shaft 328 and cam follower wheel 330 are spring biased at 336 such that cam follower wheel 330 is maintained in contact with the outer surface of cam mechanism 322 at all times, as cam mechanism 322 rotates.

In operation, as flower petal shaped cam mechanism 322 is rotated by activation of prime mover 220, cam follower wheel 330 remains in contact with the outer surface of cam mechanism 322, thus horizontally and reciprocally moving drive shaft 328 as cam follower wheel 330 advances between the peak surface points 324 and dwell surface points 326 of cam mechanism 322. This horizontal and reciprocal motion of drive shaft 328 reciprocally moves flexible barrier 208 between its original position 338 (dotted) and its distended position 340 across drip tube 206, as seen in FIG. 10. Referring to FIG. 6A, for example, flexible barrier 208 in the embodiment of FIG. 10 may also be configured to extend across a rigid barrier housing 202, where rigid barrier housing 202 is located adjacent the distal end of drip tube 206. As described with regard to the previous embodiments of the present invention, reciprocal movement of flexible bladder 208 in the embodiment illustrated in FIG. 10 provides alternate vacuum force and the relief of vacuum force in drip tube 206 for the purpose of expressing breast milk from the user, and conveying the breast milk to reservoir 12, as explained previously.

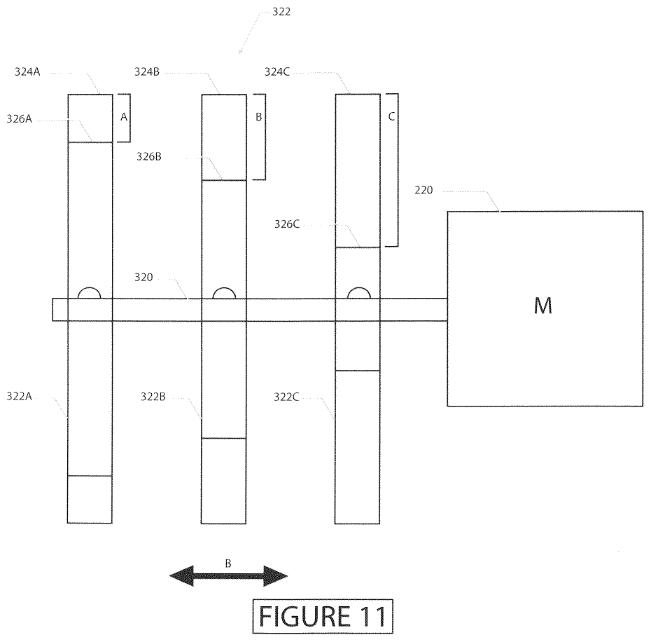

FIG. 11 is a schematic detail side view of an arrangement of multiple petal-shaped cam mechanisms 322A, 322B and 322C, similar to cam mechanism 322 in FIG. 10 but with varying radial distances on each cam mechanism 322 between peak surface points 324 and dwell surface points 326. This structure will provide varying lengths of movement, or “throw” of drive shaft 328 illustrated in FIG. 10. In the embodiment of FIG. 11, each of petal-shaped cam mechanisms 322A, B and C are slidably mounted on shaft 320 in the direction shown by arrow B, while each petal-shaped cam mechanism 322A, B and C is rotated when shaft 320 is rotated by prime mover 220. In this manner, cam follower wheel 330 and drive shaft 328 will be aligned with the cam mechanism 322 selected by the user.

Upon the shifting in direction B of petal-shaped cam mechanisms 322A, B and C, cam follower wheel 330 of drive shaft 328 will align with and come into contact with the dwell surface point 326 of the respective cam mechanism 322A, B or C aligned with cam follower wheel 330 as selected by the user. As illustrated in FIG. 11, the distances A, B and C are the varying distances between the peak surface points 324A, B and C and the dwell surface points 326A, B and C of each cam mechanism 322A, B and C, respectively. Referring to FIGS. 10 and 11, petal-shaped cam mechanism 322 is rotated by shaft 320 and prime mover 220. The distance drive shaft 328 travels in a linear axial direction (A, B, C-FIG. 11) will vary depending upon which cam mechanism 322A, B or C shaft 328 is aligned with. Thus, depending upon which cam mechanism 322 is aligned with shaft 320, the distance flexible barrier 208 (FIG. 10) travels between its original position 338 and distended position 340 can be varied by the user to increase or decrease the degree of vacuum force applied to the nipple inserted in drip tube 206.

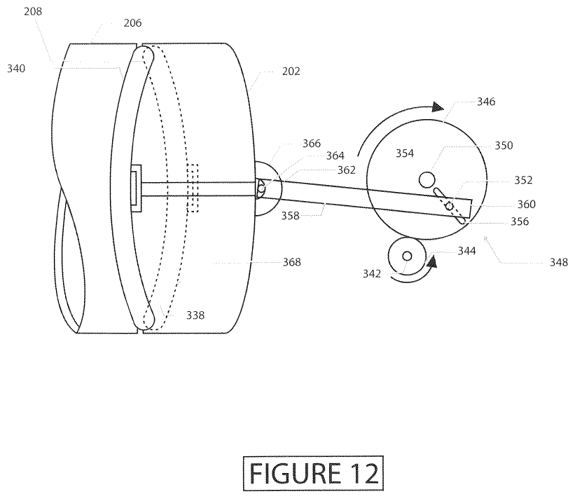

FIG. 12 schematically illustrates a further embodiment of the present invention incorporating another type of rotatory-motion-to-reciprocal-motion mechanism for moving flexible barrier 208 between its original position 338 to its distended position 340 (FIG. 10). In the embodiment of FIG. 12, a shaft 342 is rotated by prime mover 220 (FIG. 9). A relatively small pinion gear 344 is attached to shaft 342, and pinion gear 344 meshes with relatively larger spur gear 346 to create a gear reduction system 348. Spur gear 346 rotates about shaft 350, as seen in FIG. 12. An off-center pin 352 is slidably mounted on spur gear 346, with off-center pin 352 extending outward from side surface 354 of spur gear 346. Off-center pin 352 extends into elongated slot 356, where off-center pin 352 is adjustable radially in slot 356 for purposes to be explained. A locking mechanism, not shown, has one portion attached to one end of off-center pin 352, and a second portion mounted on a second side (not shown) of spur gear 346. By adjusting the locking mechanism, the radial position off-center of pin 352 in elongated slot 356 can be changed and fixed by the user.

Rotatably attached to off-center pin 352 is a drive link 358. A first end 360 of drive link 358 is rotatably mounted on off-center pin 352, and a second end 362 of drive link 358 is attached by a connector 364 to a flange 366 attached to one surface 368 of flexible barrier 208. As shown in the embodiment of FIG. 6A, flexible barrier 208 of FIG. 12 is located in a rigid barrier housing 202, and the rigid barrier housing 202 is located adjacent the distal end of drip tube 206.

In operation of the embodiment of FIG. 12, the user first adjusts the locking mechanism (not shown) to attach off-center pin 352 to spur gear 346 at a desired position. The prime mover attached to shaft 342 and pinion gear 344 is actuated, rotating pinion gear 344 and spur gear 346 through gear reduction system 348. As spur gear 346 rotates about shaft 350, the horizontal and vertical positions of off-center pin 352 (as seen in FIG. 12) change, moving second end 362 of drive link 358 to reciprocally move in a horizontal path. As the second end 362 of drive link 358 moves horizontally, flexible barrier 208 linearly reciprocally moves between its initial position 338 and its distended position 340, alternately creating a vacuum force and the relief of vacuum force in drip tube 206. As described in relation to the previously described embodiments, the application of an alternating vacuum force and relief of the vacuum force in drip tube 206 causes breast milk to be expressed from the user's breast to flow into drip tube 206, and ultimately to flow into reservoir 12.

With reference to the embodiment of FIG. 12, if the user determines that the vacuum force in drip tube 206 is too strong or too weak to express the desired amount of milk, the position of off-center pin 352 in elongated slot 356 can be adjusted to a different position in the elongated slot. This change of position of off-center pin 352 in elongated slot 356 will increase or decrease the horizontal distance, or “throw” of drive link 358, resulting in increasing or decreasing the vacuum force applied to the user's nipple in drip tube 206. The position of off-center pin 352 in elongated slot 356 can be adjusted until the user determines a comfortable and desirable application of vacuum force in drip tube 206.

In each of the embodiments of FIGS. 6A through 12, the electrical power source, prime mover, controller and mechanical drive assembly are all suitably isolated by the use of suitable walls and seals (not shown) from contact with breast milk as the milk is collected and rises in reservoir 12.

In each of the embodiments described above, the flexible barrier of the illustrated breast pump is moved from its original position to its distended position and back by several rotary-motion-to-reciprocal-motion mechanisms. Several of these rotary-motion-to-reciprocal-motion mechanisms per se may be known in the art, however the prior art does not disclose the combination of a rotary-motion-to-reciprocal-motion mechanism to operatively or directly drive a flexible barrier in a wearable breast milk collection device.

The foregoing description of illustrated embodiments of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

We claim:

1. A breast milk collection device, comprising:
an adaptor adapted at a first wide end to receive a woman's breast therein, a second narrow end of said adaptor communicating with a reservoir, said reservoir having an outer perimeter, the reservoir adapted to receive breast milk from said adaptor;
said second narrow end of said adaptor having a first aperture, a hollow drip tube communicating with the first aperture, the hollow drip tube having a second aperture through which the hollow drip tube communicates with an internal volume of said reservoir, a one-way valve element communicating with said hollow drip tube and with said internal volume preventing fluid in said reservoir from backflowing into said hollow drip tube;
a rigid barrier housing located within the outer perimeter of said reservoir;
a flexible barrier disposed across a hollow chamber formed in said rigid barrier housing, the flexible barrier creating a hermetic seal between said rigid barrier housing and said hollow chamber formed in said rigid barrier housing, said hollow chamber fluidly communicating with an interior of said hollow drip tube;
a drive assembly including a reciprocally driven member, the reciprocally driven member in operative contact with a first surface of said flexible barrier, said drive assembly reciprocally driving said flexible barrier to a first position in said hollow chamber of said rigid barrier housing during a first position of said reciprocally driven member, said flexible barrier returning to a second position during a second position of said reciprocally driven member, said reciprocally driven member reciprocally driven by a power source;
movement of said flexible barrier between said first position and said second position in said hollow chamber of said rigid barrier housing providing an alternating source of vacuum pressure and relief of vacuum pressure in said hollow drip tube.

2. The breast milk collection device of claim 1, wherein:
said drive assembly comprises a rotating-motion-to-reciprocal-motion mechanical assembly extending between said power source and said first surface of said flexible barrier.

3. The breast milk collection device of claim 2, wherein:
said rotating-motion-to-reciprocal-motion mechanical assembly comprises at least one rotatable off-center cam in operative contact with said first surface of said flexible barrier, said cam rotatably driven by said power source at one of a predetermined variable speed of rotation.

4. The breast milk collection device of claim 3, wherein:
said rotating-motion-to-reciprocal-motion mechanical assembly is located one of inside said perimeter of said reservoir and outside said perimeter of said reservoir.

5. The breast milk collection device of claim 1,
said hollow chamber formed adjacent to one end of said hollow drip tube.

6. The breast milk collection device of claim 1, wherein,
said flexible barrier is inflatable and deflatable with respect to said hollow chamber formed in said rigid barrier housing.

7. The breast milk collection device of claim 3,
a surface of said rotatable off-center cam in operative contact with said first surface of said flexible barrier.

8. The breast milk collection device of claim 1, wherein:
said power source is located within said outer perimeter of said reservoir.

9. The breast milk collection device of claim 1, wherein said power source is removably located within said outer perimeter of said reservoir.

10. The breast milk collection device of claim 1, wherein said drive assembly is removably located within said outer perimeter of said reservoir.

11. The breast milk collection device of claim 1, wherein:
said drive assembly comprises a gear reduction system operatively disposed between said power source and said reciprocally driven member.

12. The breast milk collection device of claim 1, wherein:
said hollow drip tube is connected to said rigid barrier housing.

13. The breast milk collection device of claim 1, wherein:
said power source includes a rechargeable battery.

14. The breast milk collection device of claim 13, wherein:
said rechargeable battery is rechargeable by one of a near-field wireless power transfer system and an inductive system.

15. The breast milk collection device of claim 1, comprising:
a controller operatively connected to said power source,
said controller adapted to be adjustable by a user, said controller varying operation of the drive assembly and movement of the flexible barrier.

16. The breast milk collection device of claim 3,
said reciprocally driven member including a bearing plate disposed between a surface of said rotatable off-center cam and said first surface of said flexible barrier.

17. The breast milk collection device of claim 1, said hollow chamber communicating with said hollow drip tube through an aperture disposed in said rigid barrier housing.

18. The breast milk collection device of claim 3, comprising:
a plurality of removable and replaceable off-center cams, each off-center cam having a different radial dimension from another off-center cam.

19. The breast milk collection device of claim 1,
said hollow drip tube having an aperture,
said rigid barrier housing circumscribing said aperture,
said flexible barrier extending over said aperture, said flexible barrier creating said hermetic seal between rigid barrier housing and said hollow chamber formed in said rigid barrier housing.

20. The breast milk collection device of claim 19, wherein said hollow drip tube, said flexible barrier and said rigid barrier housing are in axial alignment.

21. The breast milk collection device of claim 2, wherein:
a compartment is formed on an external surface of said reservoir;
said power source and at least a portion of said drive assembly located in said compartment;
said compartment isolated from contact with breast milk in said reservoir.

22. The breast milk collection device of claim 1;
said drive assembly further including a rotatable cylindrical cam mechanism, at least one groove extending around an outer surface of said cylindrical cam mechanisms, said at least one groove extending in a circumferential and an axial direction along said outer surface of said cylindrical cam;
said reciprocally driven member including a moveable drive shaft having an engagement element removably extending into said at least one groove of said cylindrical cam mechanism;
rotation of said rotatable cylindrical cam mechanism reciprocally moving said drive member when said engagement element extends into said at least one groove.

23. The breast milk collection device of claim 22,
one end of said moveable drive shaft having a pocket adjacent said hollow rotatable cylindrical cam mechanism;
said engagement element comprising a pin adapted to enter into and be removed from said at least one groove upon manipulation of said moveable drive shaft;
a second end of said movable drive shaft operatively connected to said first surface of said flexible barrier.

24. The breast milk collection device of claim 23, said pin being spring-biased in a direction out of said pocket.

25. The breast milk collection device of claim 22 wherein:
said rotatable cylindrical cam mechanism has a hollow interior;
said drive assembly, including said power source and a gear reduction system, are operatively connected between said power source and one of an exterior surface and an interior surface of said hollow rotatable cylindrical cam mechanism;
actuation of said power source rotating said rotatable hollow cylindrical cam mechanism and imparting reciprocal motion to said reciprocally driven member when said engagement element is inserted into said at least one groove.

26. The breast milk collection device of claim 22,
said at least one groove comprising a plurality of grooves;
each of said plurality of grooves having a different length around said outer surface of said cylindrical cam.

27. The breast milk collection device of claim 25, wherein:
said drive assembly is one of located within the hollow portion of said hollow rotatable cylindrical cam mechanism and located outside of said hollow portion of said hollow rotatable cylindrical cam mechanism.

28. The breast milk collection device of claim 22, wherein:
said rotatable cylindrical cam mechanism has a solid core.

29. The breast milk collection device of claim 2, wherein said drive assembly including said reciprocally driven member comprises:
at least one petal shaped rotatable cam member having an outer surface comprising at least one peak point and at least one dwell point;
said reciprocally driven element having a first component biased into operative contact with said outer surface of said petal shaped rotatable cam member;
said reciprocally driven element having a second component in operative engagement with a surface of said flexible barrier;
rotation of said petal shaped rotatable cam member providing said alternating source of vacuum pressure and relief of vacuum pressure in said hollow drip tube.

30. The breast milk collection device of claim 2, wherein said drive assembly comprises:
a rotatable element having a variable positionable attachment pin extending off center of rotation in an axial direction from a surface of the rotatable element;
a link operatively and rotatably connecting said attachment pin to a surface of said flexible barrier, rotation of said rotatable element imparting reciprocal motion to said link, said reciprocal motion moving said flexible barrier between said first position and said second position.

31. The breast milk collection device of claim 30, wherein:
said rotatable element includes a slot, said attachment pin movable in said slot;
a locking mechanism disposed between said slot and said attachment pin, said locking mechanism alternating locking said attachment pin in a position in the slot and releasing said attachment pin for movement in said slot.
